(19) United States
(12) Patent Application Publication
(10) Pub. No.: US 2010/0184973 A1
(43) Pub. Date: Jul. 22, 2010

(54) Method for producing dioxazine derivatives

(76) Inventors: Sergii Pazenok, Solingen (DE); Uwe Stelzer, Burscheid (DE)

Correspondence Address: Baker Donelson Bearman, Caldwell & Berkowitz, PC, 555 Eleventh Street, NW, Sixth Floor, Washington, DC, 20004, US

(21) Appl. No.: 12/664,924

(22) PCT Filed: May 28, 2008
(86) PCT No.: PCT/EP08/04215

§ 371 Date: Dec. 16, 2009

(30) Foreign Application Priority Data
Jun. 19, 2007 (EP) 07011965.6

(51) Int. Cl. C07D 401/04; C07D 213/70

(52) U.S. Cl. 544/65; 546/298

(57) ABSTRACT

A process is described for preparing dioxazine derivatives of the formula (1)

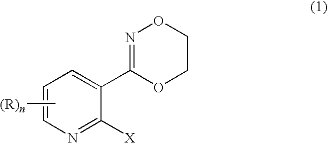

and corresponding intermediates.

The invention relates to a process for preparing dioxazine derivatives and to intermediates which are obtained during the preparation.

Dioxazines of the formula (1)

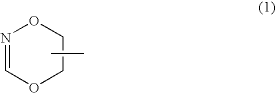

are important functional groups in a multitude of organic active compounds. For example, dioxazine rings occur in active agrochemical ingredients (cf. DE 10 2005 044 108 A1), especially in dioxazine-pyridinyl-sulfonylureas (cf. US 5,476,936). In addition, many organic pigments contain dioxazine rings (cf. DE 10 2005 063 360 A1).

The synthesis of dioxazine derivatives generally proceeds via the reaction of appropriate carboxylic esters with hydroxylamine and subsequent reaction with dibromoethane. This reaction sequence is illustrated in the following reaction equation, for example, for nicotinic esters according to U.S. Pat. No. 5,476,936:

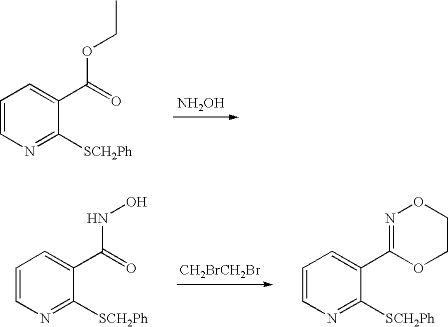

The low yield of 21% for the above-described reaction and the use of the highly toxic and environmentally damaging dibromoethane make the implementation of such a process unattractive and expensive.

There is therefore a need for an inexpensive and environmentally friendly process for preparing dioxazine derivatives, which provides the desired compounds with good yield and high purity.

It is thus an object of the present invention to provide a process for preparing dioxazine derivatives, which preferably proceeds with good yields and in which the use of highly toxic and environmentally damaging substances, especially dibromoethane, can preferably be dispensed with. The desired target compounds should preferably be obtained inexpensively and with high purity.

The above-described object is achieved in accordance with the invention by a process for preparing dioxazine derivatives of the formula (1)

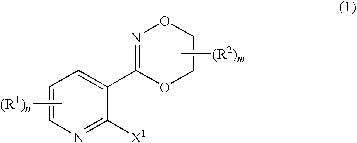

in which the individual substituents are defined as follows:
X¹ is fluorine, chlorine, bromine, iodine, SCN, or S—R³, where
   R³ is hydrogen;
      optionally substituted C₁-C₆-alkyl;
      optionally substituted C₃-C₆-cycloalkyl;
      —(CH₂)ᵣ—C₆H₅where r=an integer from 0 to 6, where the alkyl radical —(CH₂)ᵣ— may optionally be substituted; or

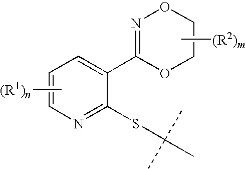

where the substituents R¹ and R² and the indices n and m have the same definitions as in the formula (1);
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2;
R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-Alkyl)carbonyl or C₃-C₆-trialkylsilyl; and
m is an integer from 0 to 4.

The process according to the invention comprises preparing the dioxazine derivatives of the formula (1) by a ring closure proceeding from a compound of the formula (2) (process step (1)):

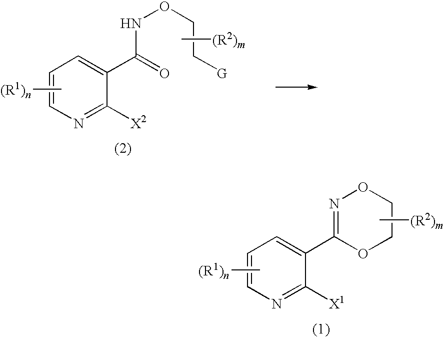

where the substituents R¹, R² and X¹ and the indices n and m are each as defined above; G is a leaving group selected from the group consisting of fluorine, chlorine, bromine, iodine, —OSO₂—CH₃, —O—SO₂CF₃, —O—SO₂—Ph and —O—SO₂—C₆H₄-Me, and X² is fluorine, chlorine, bromine, iodine, SON, or S—R³, where R³ is hydrogen; optionally substituted C₁-C₆-alkyl; optionally substituted C₃-C₆-cycloalkyl; (CH₂)ᵣ—C₆H₅ where r=0 to 6, where the alkyl radical —(CH₂)ᵣ— may optionally be substituted; or is the

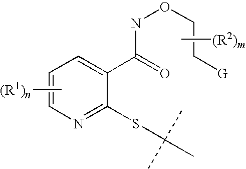

radical, where the substituents R¹ and R² and the indices m and n in the radical each have the same definitions as in the formula (1).

Compounds of the formula (2) where G=Cl are obtainable proceeding from compounds of the formula (3) by chlorination, for example with thionyl chloride (process step (2a)):

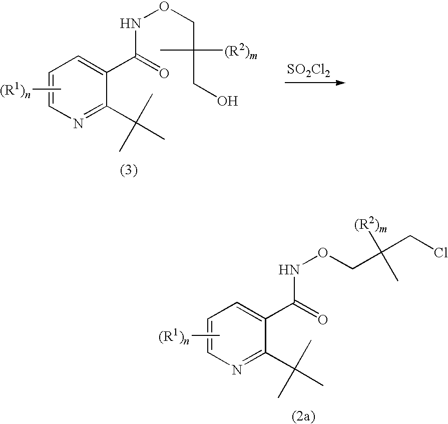

Compounds of the formula (2) where G=Br are obtainable proceeding from compounds of the formula (3) by bromination (process step (2b)):

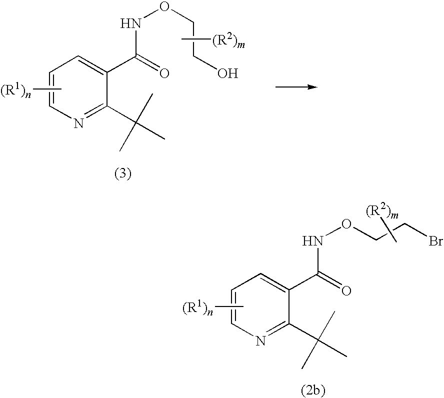

Compounds of the formula (2) where G=—O—SO₂CH₃, G=—O—SO₂—Ph or G=—O—SO₂—C₆H₄—CH₃ are obtainable proceeding from compounds of the formula (3) by reaction with CH₃SO₂Cl or PhSO₂Cl (process step (2c)):

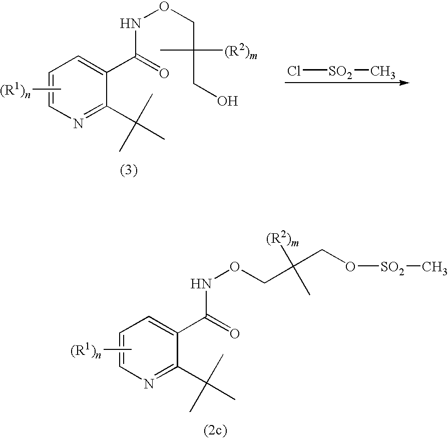

Compounds of the formula (2) where G=—O—SO₂CF₃ are obtainable from compounds of the formula (3) by reaction with (CF₃SO₂)₂O (process step (2d)):

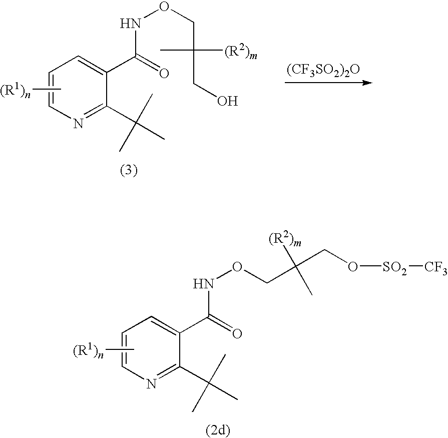

Compounds of the formula (2) where G=F are obtainable proceeding from compounds of the formula (3) by reaction with (CH₃)₂NSF₃, Deoxofluor®, the Yarovenko or Ishikawa reagent (process step (2e)):

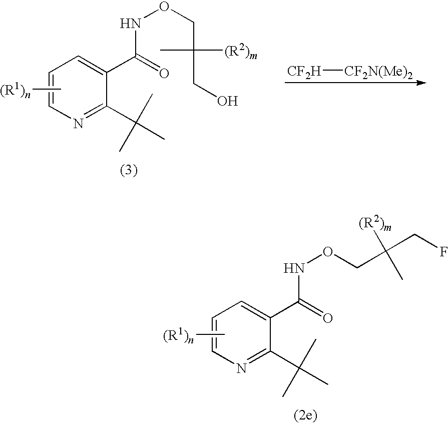

Compounds of the formula (2) where G=I are obtainable proceeding from compounds of the formula (3) by reaction with phosphorus/iodine (P/I₂) or with CH₃SO₂Cl/Kl (process step (2f)):

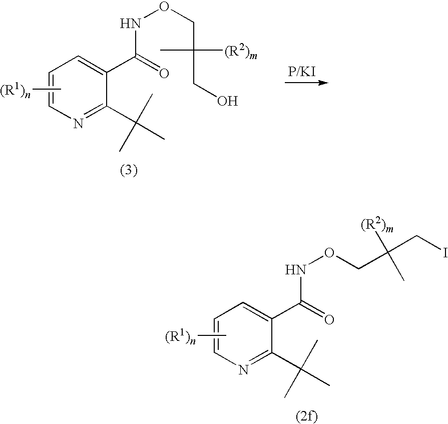

In the above-described processes for compounds (2a) to (2g) the radicals in the ortho position to the pyridine nitrogen, i.e. the substituents between pyridine nitrogen and amide/dioxazine substituent, are not defined in detail, it being possible in the case of dimer structures for the transformation on the hydroxyl group to occur twice.

Compounds of the formula (3) are known from the prior art; cf. the European patent application EP 07011966.4, filed at the same time, to Bayer CropScience AG with the title “Nicotinamide derivates and processes for preparation thereof”.

In one embodiment of the present invention, the process according to the invention comprises both process steps (1) and (2), i.e. the process in this embodiment is characterized overall by the following reaction sequence:

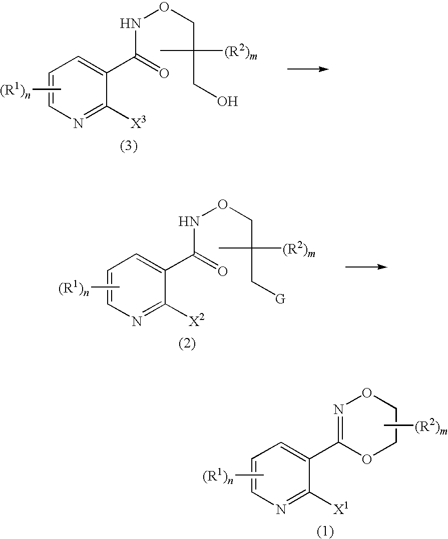

where the individual substituents each have the above definitions and X³ is fluorine, chlorine, bromine, iodine, SCN, or S—R³, where R³ is hydrogen; optionally substituted C₁-C₆-alkyl; optionally substituted C_{3—}C_{6—}cycloalkyl; —(CH₂)ᵣ—C₆H₅where r=0 to 6, where the alkyl radical —(CH₂)ᵣ— may optionally be substituted; or is the

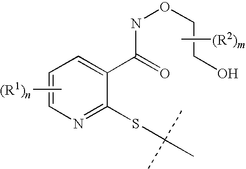

radical, where the substituents R¹ and R² and the indices m and n in the radical each have the same definitions as in the formula (1).

In the context of the present invention, the substituents of the compounds of the formulae (1) to (3) are preferably each defined as follows:
X¹, X², X³ are chlorine, S—R³, where
   R³ is optionally substituted C₁-C₆-alkyl; optionally substituted C₃-C₆-cycloalkyl;
   —(CH₂)ᵣ—C₆H₅ where r=0 to 4, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylamino-carbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² in each case is independently optionally singly or multiply, identically or differently substituted C₁-C₄-alkyl, C₃-C₆-cycloalkyl, where the substituents may each independently be selected from halogen, cyano, nitro, hydroxyl, C₁-C₄-alkoxy, C₁-C₄-haloalkoxy, C₁-C₄-alkylthio, C₁-C₄-alkylsulfinyl and C₁-C₄-alkylsulfonyl;
m is an integer from 0 to 2; and
G is fluorine, chlorine, bromine and iodine.

In addition, particular preference is given to the following definitions of the substituents of the compounds of the formulae (1) to (3):
X¹, X², X³ are chlorine, S—R³, where
   R³ is optionally substituted C₁-C₆-alkyl;
      optionally substituted C₃-C₆-cycloalkyl;
      —(CH₂)ᵣ—C₆H₅ where r=0 to 2, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylamino-carbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² in each case is independently optionally singly or multiply, identically or differently substituted C₁-C₄-alkyl, where the substituents may each independently be selected from halogen, cyano, nitro, C₁-C₄-alkoxy, C₁-C₄-haloalkoxy;
m is 0 or 1; and
G is chlorine.

In addition, the following definitions of the substituents of the compounds of the formulae (1) to (3) are especially preferred:
X¹, X², X³ are S—CH₂—C₆H₅;
n is 0;
m is 0; and
G is chlorine.

Process step (1):

The process according to the invention is characterized by the process step (1) of ring closure of the compound of the formula (2) to give the compound of the formula (1).

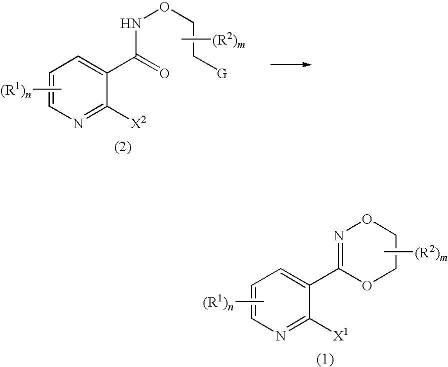

In principle, there exist several means of performing process step (1):

for instance, in a first configuration of process step (1), it is possible to perform the reaction with ring closure in the presence of bases.

The bases used in this case may be either organic or inorganic bases. Preference is given to using inorganic bases, for example LiOH, NaOH, KOH, Ca(OH)₂, Ba(OH)₂, Li₂CO₃, K₂CO₃, Na₂CO₃, NaHCO₃, or organic bases such as amines (for example, preferably triethylamine, diethylisopropylamine), Bu₄NOH, piperidine, morpholine, pyridines, alkylpyridines and DBU. Particular preference is given to using inorganic bases, for example, LiOH, NaOH, KOH, Ca(OH)₂, Ba(OH)₂, Li₂CO₃, K₂CO₃, Na₂CO₃ and NaHCO₃. Very particular preference is given to using LiOH, NaOH, KOH, K₂CO₃, Na₂CO₃, NaHCO₃.

When a base is used in process step (1) of the process according to the invention, the amount thereof is preferably 0.6 mol to 4.0 molar equivalents, more preferably 1 to 3 molar equivalents, especially 1.2 to 2.5 molar equivalents, based in each case on the compound of the formula (2).

Process step (1) is generally performed in the presence of a solvent. Process step (1) can be performed either in water or in the presence of an inert organic solvent, preferably of a polar aprotic solvent. Examples of organic solvents which can be used in the context of the present invention are aromatic or aliphatic solvents such as benzene, toluene, xylene, mesitylene, hexane, heptane, octane, cyclohexane; aliphatic and aromatic halohydrogens such as methylene chloride, dichloroethane, chloroform, carbon tetrachloride, chlorobenzene, dichlorobenzene; ethers, such as diethyl ether, dibutyl ether, diisobutyl ether, methyl tert-butyl ether, isopropyl ethyl ether, tetrahydrofuran and dioxane; and also dimethyl sulfoxide and acid amide derivatives such as N,N-dimethylformamide, N,N-dimethylacetamide and N-methyl-2-pyrrolidone; and also carboxylic esters such as ethyl acetate, or else dioxane, diglyme or dimethylglycol; nitriles such as methylnitrile, butylnitrile or phenylnitrile. Particular preference is given to toluene, xylene, dichlorobenzene, chlorobenzene, ethyl acetate, dichloroethane, N,N-dimethylformamide, N,N-dimethylacetamide and water. Especially preferred are the solvents N,N-dimethylformamide, N,N-dimethylacetamide, ethyl acetate, dichloroethane and water. However, the present invention is not limited to the solvents specified by way of example above.

The reaction temperature at which the ring closure reaction in process step (1) can be performed may vary within wide ranges. For example, the ring closure reaction can be performed at a temperature of 20 to 100° C., preferably 20 to 70° C.

Process step (1) of the process according to the invention is generally performed under standard pressure. However, it is also possible to work under elevated pressure or reduced pressure—generally between 0.1 bar and 10 bar .

In addition, it is possible in process step (1) of the present invention to use, as a reactant for the ring closure reaction, a compound which has been obtained by process stage (2), i.e., for example, by a chlorination, bromination, fluorination or mesylation. In this case, the intermediate—the compound of the formula (2)—can be used immediately as obtained in process stage (2).

When it is intended in the context of the present invention that the compound of the formula (2) is not isolated, the introduction of the leaving group G in process stage (2) and the subsequent ring closure reaction of process stage (1) can be performed as what is known as a one-pot reaction.

In this case, and hence in a second configuration of process step (1), it is possible to configure process step (1) as a one-pot reaction together with process step (2). In this configuration, it is possible to dispense with the addition of base in process step (1), which, however, somewhat lowers the overall yield over the two process stages.

The product of process step (1) can be purified by means of process operations known to those skilled in the art, for example crystallization or chromatography, although the purity of the crude product is already sufficient for use in subsequent reactions.

Process step (2):

Process step (2) comprises the transformation of the hydroxyl function of the compound of the formula (3) according to the following reaction equation to a leaving group selected from the group consisting of fluorine, chlorine, bromine, iodine, —OSO₂—CH₃, —O—SO₂CF₃, —O—SO₂—Ph and —O—SO₂—C₆H₄-Me:

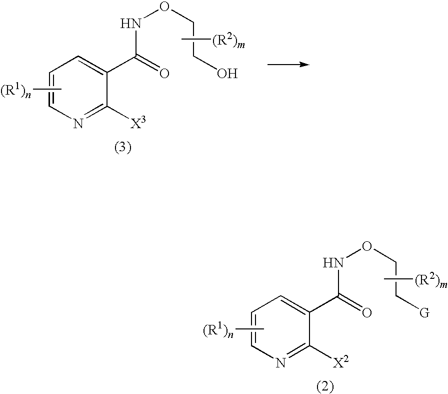

With regard to the individual R¹, R², X² and X³ radicals and the indices m and n, reference is made to the corresponding definitions.

The compounds of the formula (2) obtained in this process step (2) can be used as a reactant in process step (1) of the process according to the invention, though it is possible to work up the compound of the formula (2) after process step (2), i.e. to use it in isolated and optionally purified form, or else to use it in unpurified form (one-pot reaction).

Depending on the selection of the leaving group, there are several means of performing the transformation in process step (2).

If the leaving group is chlorine, which is also preferred in the context of the present invention, it is possible to use any desired chlorinating agent to chlorinate the compound of the formula (3). Useful examples include thionyl chloride (SOCl₂), phosphoryl chloride (POCl₃), phosgene, diphosgene and oxalyl chloride ((COCl)₂). Particularly preferred among these are thionyl chloride (SOCl₂), phosgene and oxalyl chloride ((COCl)₂).

The amount of chlorinating agent used may vary within wide ranges. For example, the amount of chlorinating agent used for process step (2) is 0.8 to 3 molar equivalents, more preferably 1 to 2.5 molar equivalents, especially 1.1 to 1.8 molar equivalents, based in each case on the amount of compounds of the formula (3). If the leaving group is bromine, the compound of the formula (3) can be brominated using any desired brominating agents. Useful examples include phosphorus tribromide (PBr₃) or phosphoryl bromide (POBr₃).

The amount of brominating agent used may vary within wide ranges. For example, the amount of brominating agent used for process step (2) is 0.8 to 3 molar equivalents, more preferably 1 to 2.5 molar equivalents, especially 1.1 to 1.8 molar equivalents, based in each case on the amount of compounds of the formula (3).

If the leaving group is fluorine, the compound of the formula (3) can be fluorinated using any desired fluorinating agent. Useful examples include (CH₃)₂NSF₃ (DAST), Deoxofluor®, the Yarovenko or Ishikawa reagent (ClCFH—CF₂—N(C₂H₅)₂).

The amount of fluorinating agent used may vary within wide ranges. For example, the amount of fluorinating agent used for process step (2) is 0.8 to 3 molar equivalents, more preferably 1 to 1.5 molar equivalents, especially 1 to 1.3 molar equivalents, based in each case on the amount of compounds of the formula (3).

If the leaving group is iodine, the compound of the formula (3) can be iodinated using any desired iodinating agents. Useful examples include I₂/P or CH₃SO₂Cl/KI.

The amount of iodinating agent used may vary within wide ranges. For example, the amount of iodinating agent used for process step (2) is 0.8 to 2 molar equivalents, more preferably 1 to 1.5 molar equivalents, especially 1 to 1.2 molar equivalents, based in each case on the amount of compounds of the formula (3).

If the leaving group is —O—SO₂—CH₃, —OSO₂—Ph, —OSO₂—C₆H₄—CH₃, the leaving group can be introduced into the compound of the formula (3) using methanesulfonyl chloride (CH₃—SO₂—Cl), phenyl sulfochloride (PhSO₂Cl) or tolyl sulfochloride CH₃—C₆H₄SO₂—Cl.

The amount of methanesulfonyl chloride (CH₃—SO₂—Cl), phenyl sulfochloride (PhSO₂Cl) or tolyl sulfochloride (CH₃—C₆H₄SO₂Cl) used may vary within wide ranges. For example, the amount of reagents used for process step (2) is 0.8 to 3 molar equivalents, more preferably 1 to 2.5 molar equivalents, especially 1 to 1.5 molar equivalents, based in each case on the amount of compounds of the formula (3).

If the leaving group is —O—SO₂—CF₃, the leaving group can be introduced into the compound of the formula (3) using trifluoromethylsulfonic anhydride (CF₃—SO₂)₂O.

The amount of trifluoromethylsulfonic anhydride used may vary within wide ranges. For example, the amount of trifluoromethylsulfonic anhydride used for process step (2) is 0.8 to 2.5 molar equivalents, more preferably 1 to 2 molar equivalents, especially 1 to 1.5 molar equivalents, based in each case on the amount of compounds of the formula (3).

Process step (2) is generally performed in the presence of a solvent. The solvents used may, for example, be organic solvents. Examples of organic solvents are aromatic or aliphatic solvents such as benzene, toluene, xylene, mesitylene, hexane, heptane, octane, cyclohexane; aliphatic and aromatic halohydrogens such as methylene chloride, dichloroethane, chloroform, carbon tetrachloride, chlorobenzene, dichlorobenzene; acid amide derivatives such as N,N-dimethylformamide, N,N-dimethylacetamide and N-methyl-2-pyrrolidone; and also carboxylic esters such as ethyl acetate; or else dioxane, diglyme, dimethylglycol or THF; nitriles such as methylnitrile, butylnitrile or phenylnitrile. Particular preference is given to toluene, xylene, dichlorobenzene, chlorobenzene or ethyl acetate. Among these, the following solvents are particularly preferred: methylene chloride, dichloroethane, ethyl acetate, N,N-dimethylformamide, N,N-dimethylacetamide and N-methyl-2-pyrrolidone.

The reaction temperature at which the reaction in process step (2) can be performed may vary within wide ranges. For example, the ring closure reaction can be performed at a temperature of 10 to 100° C., preferably 20 to 80° C. The reaction temperature depends on the reactivity of the individual compounds.

Process step (2) of the process according to the invention is generally performed under standard pressure. However, it is also possible to work under elevated pressure or reduced pressure—generally between 0.1 bar and 10 bar.

The product of process step (2) can be purified by means of process operations known to those skilled in the art, for example crystallization or chromatography, although the purity of the crude product is already sufficient to be used in the subsequent reaction of process step (1).

The process according to the invention affords the desired dioxazine derivatives in high yield and purity. The process according to the invention can be performed in a simple manner and more particularly without use of environmentally damaging reagents. Owing to the possibility of a one-pot reaction, the process is inexpensive; corresponding workups of the intermediate and of the target compound can be dispensed with.

Furthermore, the compounds of the formula (2) with G selected from the group consisting of fluorine, chlorine, bromine, iodine, —OSO₂—CH₃, —O—SO₂CF₃, —O—SO₂—Ph and —O—SO₂—C₆H₄-Me are novel.

The present invention therefore further provides compounds of the formula (2)

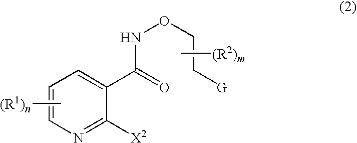

in which the individual substituents are defined as follows:
G is fluorine, chlorine, bromine, iodine, —OSO₂—CH₃, —O—SO₂CF₃, —O—SO₂—Ph and —O—SO₂—C₆H₄-Me;
X² is fluorine, chlorine, bromine, iodine, SCN, or S—R³, where
   R³ is hydrogen; optionally substituted C₁-C₆-alkyl; optionally substituted C₃-C₆-cycloalkyl; —(CH₂)ᵣ—C₆H₅ where r=0 to 6, where the alkyl radical —(CH₂)ᵣ— may optionally be substituted; or is the radical,

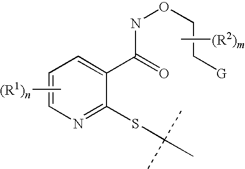

R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2;
R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-Alkyl)carbonyl or C₃-C₆-trialkylsilyl; and
m is an integer from 0 to 4.

In a preferred embodiment, the substituents are defined as follows:
G is fluorine, chlorine, bromine and iodine.
X² is chlorine, S—R³, where
   R³ is optionally substituted C₁-C₆-alkyl;
      optionally substituted C₃-C₆-cycloalkyl;
      —(CH₂)ᵣ—C₆H₅where r=0 to 4, where the alkyl radical —(CH₂)— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² in each case is independently optionally singly or multiply, identically or differently substituted C₁-C₄-alkyl, C₃-C₆-cycloalkyl, where the substituents may each independently be selected from halogen, cyano, nitro, hydroxyl, C₁-C₄-alkoxy, C₁-C₄-haloalkoxy, C₁-C₄-alkylthio, C₁-C₄-alkylsulfinyl and C₁-C₄-alkylsulfonyl; and
m is an integer from 0 to 2.

In addition, the following definitions of the substituents of the compounds of the formula (2) are particularly preferred:
G is chlorine;
X² is chlorine, S—R³, where
   R³ is optionally substituted C₁-C₆-alkyl;
      optionally substituted C₃-C₆-cycloalkyl;
      —(CH₂)ᵣ—C₆H₅where r=0 to 2, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;

1n is 0 or 1;
R² is optionally singly or multiply, identically or differently substituted C₁-C₄-alkyl, where the substituents may each independently be selected from halogen, cyano, nitro, C₁-C₄-alkoxy, C₁-C₄-haloalkoxy; and
m is 0 or 1.

In addition, the following definitions of the substituents of the compounds of the formula (2) are particularly preferred:
G is chlorine;
X is S—CH₂-C₆H₅;
n is 0;
m is 0.

These compounds can be obtained by the processes described above. The invention will now be illustrated in detail by the working example which follows, but without restricting the invention thereto.

### WORKING EXAMPLE

### Example 1

### Preparation of 2-(benzylthio)-N-(2-chloroethoxy)nicotinamide

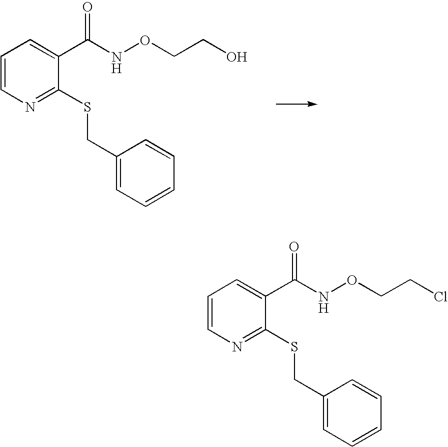

11.4 g of 2-(benzylthio)-N-(2-chloroethoxy)nicotinamide were initially charged in 50 ml of N,N-dichloromethane, and 6.6 g of thionyl chloride were added dropwise at 30° C. The reaction mixture was stirred at room temperature for 1 hour, and then the mixture was diluted with 100 ml of water. The suspension was stirred at 20° C. for 1 hour, and the precipitate was filtered off with suction and washed with water.

This gave 11.47 g, 95% of theory, of the product (m.p. 122-124° C.).

¹H NMR (DMSO-d₆): 3.81 (m, 2H), 4.15 (m, 2H), 4.4 (s, 2H), 7.2-7.4 (m, 6H), 7.8 (dd, 1 H), 8.5 (dd, 1 H).

### Example 2

### Preparation of 3[2-(benzylthio)pyridin-3-yl]-5,6-dihydro-1,4,2-dioxazine (without base)

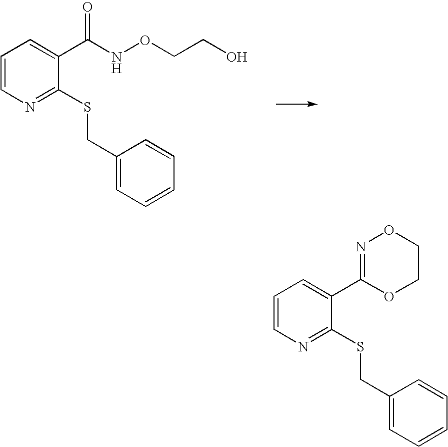

114 g of 2-(benzylthio)-N-(2-hydroxyethoxy)nicotinamide were initially charged in 500 ml of THF, and 65.8 g of thionyl chloride were added dropwise at 10° C. The reaction mixture was stirred at room temperature for 1 hour, and then the precipitate (HCl salt of the product) was filtered off and washed with water and NaHCO₃ solution and dried.

This gave approx. 65 g of the product with m.p. 63-65° C.

¹H NMR (DMSO-d₆): 4.2 (d, 2H), 4.4 (s, 2H), 4.45 (d, 2H), 7.2-7.4 (m, 6H), 7.7 (d, 1H), 8.5 (d, 1H).

### Example 3

### Preparation of 3-[2-(benzylthio)pyridin-3-yl]-5,6-dihydro-1,4,2-dioxazine (with base)

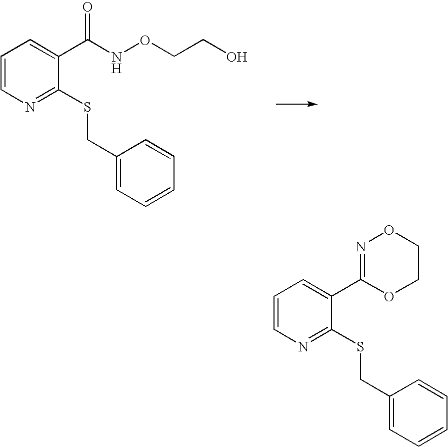

114 g of 2-(benzylthio)-N-(2-hydroxyethoxy)nicotinamide were initially charged in 500 ml of N,N-dimethylacetamide, and 65.8 g of thionyl chloride were added dropwise. The reaction mixture was stirred at room temperature for 1 hour and then 306 g of potash solution were added dropwise at room temperature. The mixture was heated to 70° C. and stirred at this temperature for approx. 1 hour. Water was added and the suspension was stirred at 20° C. for 3 hours. The precipitate was filtered off with suction and washed with water.

This gives 103 g, 95% of theory (purity 98%, 100% LC), m.p. 62-64° C.

1. A process for preparing a dioxazine derivative of the formula (1) 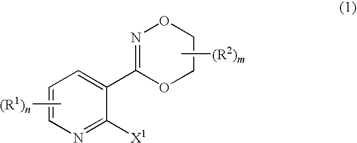
X¹ is fluorine, chlorine, bromine, iodine, SCN, or S—R³, where
R³ is hydrogen;
optionally substituted C₁-C₆-alkyl;
optionally substituted C₃-C₆-cycloalkyl;
—(CH₂)ᵣ—C₆H₅ where r=0 to 6, where the alkyl radical —CH₂)ᵣ— may optionally be substituted; or 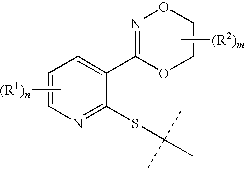
wherein
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2;
R² is in each case independently optionally singly or multiply, identically or differently substituted C₁-C₆-alkyl, C₂-C₆-alkenyl, C₂-C₆-alkynyl, C₃-C₆-cycloalkyl, where the substituents may each independently be selected from halogen, cyano, nitro, C₁-C₄-alkoxy, C₁-C₄-haloalkoxy, Cᵢ-C₄-alkylthio, C₁-C₄-alkylsulfinyl, C₁-C₄-alkylsulfonyl, (C₁-C₆-alkoxy)carbonyl, (C₁-C₆-Alkyl)carbonyl or C₃-C₆-trialkylsilyl; and
m is an integer from 0 to 4,
which comprises
preparing a dioxazine derivative of formula (1) by a ring closure proceeding from a compound of the formula (2) (process step (1)): 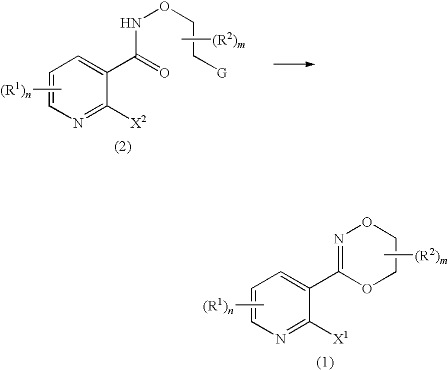
where
G is a leaving group selected from the group consisting of fluorine, chlorine, bromine, iodine, —OSO₂—CH₃, —O—SO₂CF₃, —O—SO₂—Ph and —O—SO₂—C₆H₄-Me
X² is fluorine, chlorine, bromine, iodine, SCN, or S—R³,
where R³ is hydrogen;
optionally substituted C₁-C₆-alkyl;
optionally substituted C₃-C₆-cycoalkyl;
—(CH₂)ᵣ—C₆H₅ where r=0 to 6, where the alkyl radical —(CH₂)ᵣ— may optionally be substituted; or is

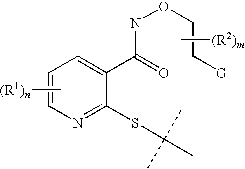

2. The process as claimed in claim 1, wherein a compound of formula (2) is obtainable by transformation of a hydroxyl function of a compound of formula (3) to a leaving group selected from the group consisting of fluorine, chlorine, bromine, iodine, —OSO₂—CH₃, —O—SO₂CF₃, —O—SO₂—Ph and —O—SO₂—C₆H₄-Me: 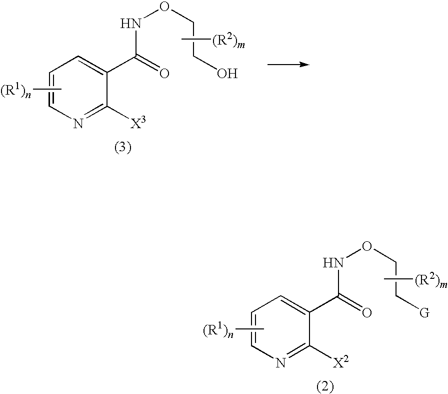
where X³ is fluorine, chlorine, bromine, iodine, SCN, or S—R³, where R³ is hydrogen; optionally substituted C₁-C₆-alkyl; optionally substituted C_{3—}C_{6—}cycloalkyl; —(CH₂)ᵣ—C₆H₅ where r=0 to 6, where the alkyl radical —(CH₂)ᵣ— may optionally be substituted; or is

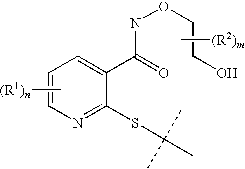

3. The process as claimed in claim 1, wherein cyclization in said process step (1) is performed in the presence of a base.

4. The process as claimed in of claim 1, wherein the reaction in said process step (1) is performed at a temperature of 20 to 100° C.

5. The process as claimed in claim 2, wherein the leaving group is chlorine and, a chlorination is performed by a chlorinating agent selected from the group consisting of thionyl chloride (SOCl₂), phosphoryl chloride (POCl₃), phosgene, diphosgene and oxalyl chloride ((COCl)₂).

6. The process as claimed in claim 2, wherein at least a portion of the process is performed at a temperature of 10 to 100° C.

7. The process as claimed in claim 2, wherein said process comprises a one-pot reaction without isolating the compound of formula (2).

8. The process as claimed in claim 7, wherein the one-pot reaction is performed without adding a base.

9. A compound of formula (2) 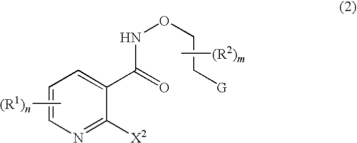
in which
G is fluorine, chlorine, bromine, iodine, —OSO₂—CH₃, —O—SO₂CF₃, —O—SO₂—Ph and/or —O—SO₂—C₆H₄-Me;
X² is fluorine, chlorine, bromine, iodine, SCN, or S⁻R³, where
R³ is hydrogen;
optionally substituted C₁-C₆-alkyl;
optionally substituted C₃-C₆-cycloalkyl;
—(CH₂)ᵣ—C₆H₅ where r=0 to 6, where the alkyl radical —(CH₂)ᵣ— may optionally be substituted; or is 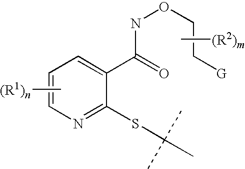
where
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is an integer from 0 to 2;
R² is in each case independently optionally singly or multiply, identically or differently substituted Cᵢ-C₆-alkyl, C₂-C₆-alkenyl, C₂-C₆-alkynyl, C₃-C₆-cycloalkyl, where the sub stituents may each independently be selected from halogen, cyano, nitro, C₁-C₄-alkoxy, C₁-C₄-haloalkoxy, C₁-C₄-alkylthio, C₁-C₄-alkylsulfinyl, C₁-C₄-alkylsulfonyl, (C₁-C₆-alkoxy)carbonyl, (C₁-C₆-Alkyl)carbonyl or C₃-C₆-trialkylsilyl; and
m is an integer from 0 to 4.

10. A compound of formula (2) as claimed in claim 9, where
G is fluorine, chlorine, bromine and/or iodine.
X² is S—R³, where
R³ is optionally substituted C₁-C₆-alkyl;
optionally substituted C₃-C₆-cycloalkyl;
—(CH₂)ᵣ—C₆H₅ where r=0 to 4, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² in each case is independently optionally singly or multiply, identically or differently substituted C₁-C₄-alkyl, C₃-C₆-cycloalkyl, where the substituents may each independently be selected from halogen, cyano, nitro, hydroxyl, C₁-C₄-alkoxy, C₁-C₄-haloalkoxy, C₁-C₄-alkylthio, C₁-C₄-alkylsulfinyl and C₁-C₄-alkylsulfonyl; and
m is an integer from 0 to 2.

11. A compound of the formula (2) as claimed in claim 9, where
G is chlorine;
X² is S—R³, where
R³ is optionally substituted C₁-C₆-alkyl;
optionally substituted C₃-C₆-cycloalkyl;
—(CH₂)ᵣ—C₆H₅ where r=0 to 6, where the alkyl radical —(CH₂)ᵣ— may optionally be substituted;
R¹ is halogen; cyano; thiocyanato; or in each case optionally halogen-substituted alkyl, alkenyl, alkynyl, alkoxy, alkylthio, alkylsulfinyl, alkylsulfonyl, alkylamino, alkylcarbonyl, alkoxycarbonyl, alkylaminocarbonyl, aryl, heteroaryl, cycloalkyl and heterocyclyl, where the alkyl and alkylene groups in the aforementioned radicals may each contain 1 to 6 carbon atoms, the alkenyl and alkynyl groups each 2 to 6 carbon atoms, the cycloalkyl groups each 3 to 6 carbon atoms and the aryl groups each 6 or 10 carbon atoms;
n is 0 or 1;
R² is optionally singly or multiply, identically or differently substituted C₁-C₄-alkyl, where the substituents may each independently be selected from halogen, cyano, nitro, C₁-C₄-alkoxy, C₁-C₄-haloalkoxy; and
m is 0 or 1.

12. A compound of formula (2) as claimed in claim 9, where
G is chlorine;
X² is S—CH₂-C₆H₅;
n is 0; and
m is 0.

13. A compound of formula (2) as claimed in claim 10, where
G is chlorine;
X² is S—CH₂-C₆H₅;
n is 0; and
m is 0.

14. A compound of formula (2) as claimed in claim 11, where
G is chlorine;
X² is S—CH₂-C₆H₅;
n is 0; and
m is 0.

15. The process as claimed in claim 3, wherein said process comprises_a one-pot reaction without isolating the compound of formula (2).

16. The process as claimed in claim 4, wherein said process comprises_a one-pot reaction without isolating the compound of formula (2).

17. The process as claimed in claim 5, wherein said process comprises_a one-pot reaction without isolating the compound of formula (2).

18. The process as claimed in claim 6, wherein said process comprises_a one-pot reaction without isolating the compound of formula (2).

19. The process as claimed in claim 3, wherein at least a portion of the process is performed at a temperature of 10 to 100° C.

20. The process as claimed in claim 4, wherein at least a portion of the process is performed at a temperature of 10 to 100° C.
